(19) United States
(12) Patent Application Publication
(10) Pub. No.: US 2010/0179098 A1
(43) Pub. Date: Jul. 15, 2010

(54) FUROST-5-ENE-3, 22, 26-TRIOL GLYCOSIDE COMPOUND FOR PREVENTING AND TREATMENT CANCER

(75) Inventors: Shoei-Sheng Lee, Taipei City (TW); Ming-Yang Lai, Taipei City (TW); Chien-Kuang Chen, Taipei City (TW); Chih-Chiang Wang, Taipei City (TW)

Correspondence Address: OSHA LIANG L.L.P., TWO HOUSTON CENTER, 909 FANNIN, SUITE 3500, HOUSTON, TX, 77010, US

(73) Assignee: HENKAN PHARMACEUTICAL CO., LTD., Taipei (TW)

(21) Appl. No.: 12/440,273

(22) PCT Filed: Sep. 7, 2006
(86) PCT No.: PCT/CN2006/002314

§ 371 Date: Dec. 4, 2009

(51) Int. Cl. A61K 31/7048; A61P 35/00

(52) U.S. Cl. 514/26

(57) ABSTRACT

A pharmaceutical composition for preventing and treating cancer comprising furost-5-ene-3,22,26-triol glycoside, which can be used to prevent and treat cancer by promoting apoptosis.

### FIELD OF THE INVENTION

The present invention relates to a steroidal glycoside compound for cancer prevention and treatment. In particular, the present invention relates to a furost-5-ene-3,22,26-triol glycoside for promoting apoptosis to prevent and treat cancer.

### BACKGROUND OF THE INVENTION

Cancer has always been the first among the top ten causes of death according to the statistics published by the Department of Health, Executive Yuan, Taiwan in recent years. It shows that cancer treatment still is an area that requires a lot of efforts and improvement.

At present, many approaches for treating cancer are available, and these approaches can be divided into non-surgical operation and surgical therapy. Among the non-surgical alternatives, chemotherapeutic drugs are commonly used; however, the therapeutic efficacies of these drugs are often limited and often accompanied with serious side effects in the metastatic and later stage. Thus, the chemotherapeutic effects of chemotherapy are often disappointing. Even with the newly developed anti-angiogenesis therapy, the therapeutic effect thereof is also limited.

For reasons set forth above, embodiments of the invention aim at overcoming some of the difficulties noted in prior art cancer therapies, by providing a steroidal glycoside compound with a furan structure to promote apoptosis for preventing and treating cancer.

### SUMMARY OF THE INVENTION

In accordance with the first aspect of the present invention, a pharmaceutical composition including a furost-5-ene-3,22,26-triol glycoside having an effective dosage as shown in formula I is provided.

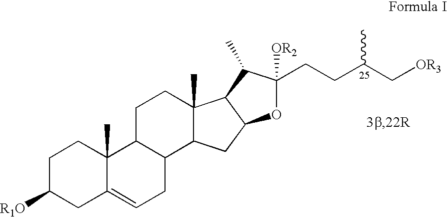

In the formula I, R₁ is one selected from the group consisting of hydrogen, a glucose, a rhamnose, a galactose, a xylose, an arabinose, a tetra-saccharide, a penta-saccharide, and a hexa-saccharide, wherein the tetra-saccharide, penta-saccharide, and hexa-saccharide are each composed of monosaccharides selected from glucose, rhamnose, galactose, xylose and arabinose. The stereo configuration at C-25 is either R (rectus) form or S (sinister) form. R₂ is hydrogen and methyl group, and R₃ is one selected from the group consisting of hydrogen, a glucose, a rhamnose, a galactose, a xylose, and an arabinose. The pharmaceutical composition further includes a pharmaceutically acceptable carrier or excipient.

Preferably, the pharmaceutical composition has the function of promoting apoptosis.

Preferably, the pharmaceutical composition has the function of preventing and treating cancer.

Preferably, the pharmaceutical composition has the function of preventing and treating liver cancer, lung cancer or colon cancer.

Preferably, the furost-5-ene-3,22,26-triol glycoside of the formula I is dichotomin, (25R)-26-O-beta-D-glucopyranosyl-22-hydroxy-5-ene-furostan-3beta, 26-diol-3-O-alpha-L-rhamnopyranosyl-(1→4)-alpha-L-rhamnopyranosyl-(1→4)-[alpha-L-rhamnopyranosyl-(1→2)]-beta-D-glucopyranoside.

Preferably, the furost-5-ene-3,22,26-triol glycoside of the formula I is 26-O-β-D-glucopyranosyl-22α-methoxy-(255)-furost-5-ene-3β,26-diol 3-O-α-L-rhamnopyranosyl-(1→4)-β-D-glucopyranoside.

Preferably, the furost-5-ene-3,22,26-triol glycoside of the formula I is 26-O-β-D-glucopyranosyl-22α-methoxy-(25R)-furost-5-ene-3β,26-diol 3-O-α-L-rhamnopyranosyl-(1→4)-β-D-glucopyranoside.

Preferably, the furost-5-ene-3,22,26-triol glycoside of the formula I is 26-O-β-D-glucopyranosyl-22α-methoxy-(25R)-furost-5-ene-3β,26-diol 3-O-α-L-rhamnopyranosyl-(1→2)-α-L-rhamnopyranosyl-(1→4)-β-D-glucopyranoside.

Preferably, the furost-5-ene-3,22,26-triol glycoside of the formula I is extracted from a water extract or a partially purified composition from a plant of the Livistona genus.

Preferably, the furost-5-ene-3,22,26-triol glycoside of the formula I is extracted form a water extract or a partially purified composition from a plant of the Asparagus genus.

In accordance with the second aspect of the present invention, a method for preparing a pharmaceutical composition is provided. The method including steps of: (a) providing one plant of the Livistona or Asparagus genus; and (b) extracting each plant with water to obtain a water extract containing a furost-5-ene-3,22,26-triol glycoside of a formula I.

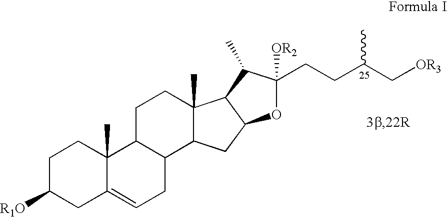

Preferably, the method further includes a step of extracting the water extract with an organic solvent.

Preferably, the organic solvent is n-butanol or ethyl acetate.

The above objectives and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 illustrates a flow chart showing the preparation of furost-5-ene-3,22,26-triol glycoside compound of the present invention;

FIGS. 2(a) and 2(b) are the apoptotic results performed with a Livistona chinensis extract of the present invention, wherein FIG. 2(a) is the experimental result of the L. chinensis extract PK-1-1, and FIG. 2(b) is that of the L. chinensis extract PK-1-2;

FIGS. 3(a) and 3(b) show microscopic photographs illustrating apoptosis of the cancer cells induced by a pure compound, dichotomin (PK-22-1), of the present invention injected into rats with tumors. FIG. 3(a) is the control group injected with phosphate buffered saline (PBS), and FIG. 3(b) is the experimental group injected with dichotomin (PK-22-1);

FIGS. 4(a) and 4(b) show electrophoresis patterns illustrating nuclear DNA fragmentation induced by dichotomin (PK-22-1) of the present invention to (a) GP7TB and (b) HepG2 cells, respectively;

FIGS. 5(a) and 5(b) show ex-vivo experimental results of cancer inhibition by (a) dichotomin (PK-22-1) and (b) compound PK-22-3 of the present invention in F344 rats grafted with GP7TB cells; and

FIG. 6 shows a high performance liquid chromatography (HPLC) pattern of the isolated compounds 5-7 of the present invention.

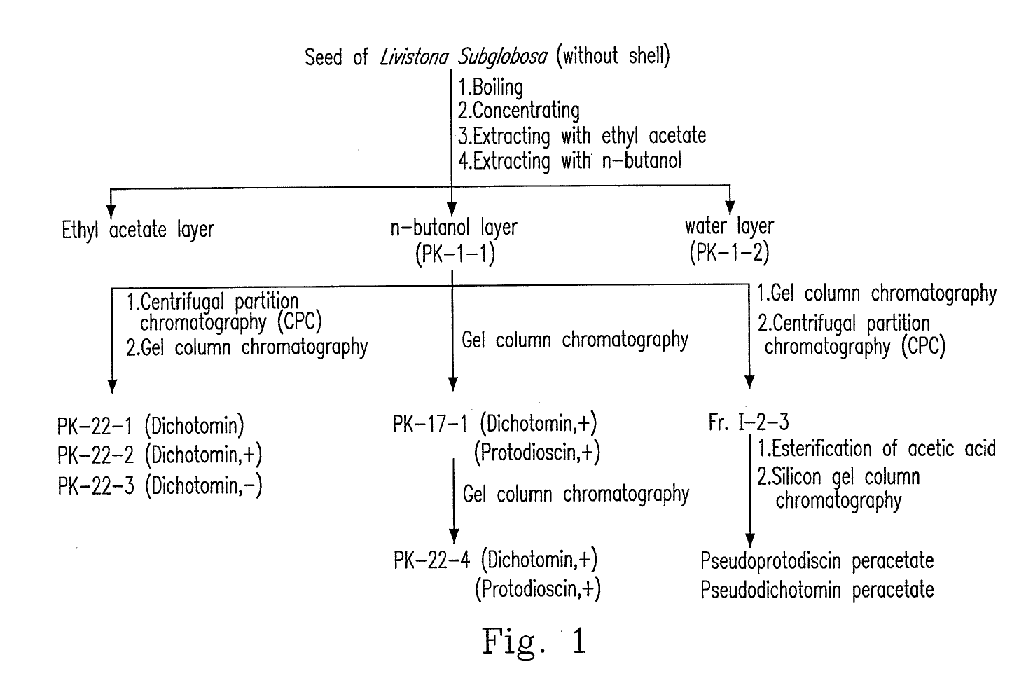

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only; it is not intended to be exhaustive or to limit the scope of the invention to the precise form disclosed.

### (1) Purification of Livistona chinensis Extract of the Prevent Invention

Two hundred grams of seeds of Livistona chinensis without shell were milled and boiled in 0.8 L of water for 2 hours to extract the active ingredients. The boiling/extraction was repeated for a total of 3 times. The extraction supernatants were centrifuged and concentrated under reduced pressure to a volume of 200 ml. The concentrated supernatant was extracted separately with ethyl acetate (200 ml, 3 times) and n-butanol (200 ml, 3 times). An n-butanol soluble product of 2.34 g (i.e. PK-1-1) and a water soluble product of 13.78 g (i.e. PK-1-2) were obtained after evaporation under vacuum. A portion of the n-butanol soluble product (i.e. PK-1-1, 1.55 g) was separated with centrifugal partition chromatography (CPC) using a solvent system of n-butanol-methanol-water (4:1:4), first with the organic phase as the mobile phase, then with the water phase as the mobile phase. Subsequent separation of a fraction from n-butanol elution on a Sephadex LH-20 column (140 ml, MeOH—H₂O 1:1) gave 18 mg of dichotomin (i.e. Compound 1, PK-22-1), together with a partially purified dichotomin fraction (i.e. PK-22-2; 21 mg) and another fraction without dichotomin (i.e. PK-22-3; 17 mg).

### (2) Preparation of the Fractions Containing Dichotomin (PK-17-1 and PK-22-4)

A portion of the n-butanol soluble product (i.e. PK-1-1, 1.03 g) was fractionated on a Sephadex LH-20 column (140 ml, MeOH—H₂O 1:1) to obtain a partially purified fraction containing dichotomin (i.e. PK-17-1; 234 mg). This partially purified fraction (203 mg) was further purified on Sephadex LH-20 to obtain another fraction containing dichotomin (i.e. PK-22-4, 36 mg).

Dichotomin (Compound 1) is an amorphous powder, [α]_{D}²⁵=−84° (c 1.0, MeOH); IR vₘₐₓ cm⁻¹ (KBr): 3437 (br s, OH), 2933 (m), 1633 (m), 1454 (m), 1388 (m), 1262 (m), 1128 (s), 1049 (s), 910 (w), 804 (w); ¹H-NMR (CD₃OD, 400 MHz): δ 1.87 (br d, J=14.2 Hz, H-1β), 1.07 (m, H-1α), 1.61 (m, H-2β), 1.91 (m, H-2α), 3.59 (m, H-3α), 2.28 (br t, J=13.0 Hz, H-4β), 2.44 (dd, J=2.8, 13.0 Hz, H-4α), 5.38 (br d, J=2.9 Hz, H-6), 1.58 (m, H-7α), 2.01 (m, H-7α), 1.66 (m, H-8), 0.95 (m, H-9), 1.53 (m, H-11β), 1.56 (m, H-11α), 1.80 (m, H-12β), 1.18 (m, H-12α), 1.13 (m, H-14), 1.27 (m, H-15β), 1.97 (m, H-15α), 4.36 (dd, J=7.4, 14.4 Hz, H-16), 1.73 (m, H-17), 0.83 (s, 3H, H-18), 1.04 (s, 3H, H-19), 2.17 (dq, J=6.2, 7.7 Hz, H-20), 1.00 (d, J=6.2 Hz, 3H, H-21), 1.58 (m, H-23), 1.81 (m, H-23), 1.15 (m, H-24), 1.59 (m, H-24), 1.74 (m, H-25), 3.72 (m, H-26), 3.39 (m, H-26), 0.94 (d, J=6.4 Hz, 3H, H-27), 3-O-glu: 4.49 (d, J=7.7 Hz, H-1′), 3.25 (m, H-2′), 3.56 (m, H-3′), 3.52 (m, H-4′), 3.30 (m, H-5′), 3.79 (dd, J=1.6, 12.4 Hz, H-6′), 3.64 (m, H-6′), rha-(1→2): 5.18 (br s, H-1″), 3.93 (m, H-2″), 3.61 (m, H-3″), 3.40 (m, H-4″), 4.11 (dq, J=6.0, 9.4 Hz, H-5″), 1.23 (d, J=6.0 Hz, 3H, H-6″), rha-(1→4)-rha-(1→4): 4.83 (buried in DOH, H-1′″), 3.77 (m, H-2′″), 3.74 (m, H-3′″), 3.53 (m, H-4′″), 4.03 (dq, J=6.2, 9.4 Hz, H-5′″), 1.28 (d, J=6.2 Hz, 3H, H-6′″), 26-O-glu: 4.23 (d, J=7.8 Hz, H-1″″), 3.18 (dd, J=7.8, 8.7 Hz, H-2″″), 3.37 (m, H-3″″), 3.34 (m, H-4″″), 3.27 (m, H-5″″), 3.85 (dd, J=1.5, 12.4 Hz, H-6″″), 3.66 (m, H-6″″), rha-(1→4)-rha-(1→4): 5.17 (br s, H-1′″″), 3.93 (m, H-2′″″), 3.66 (m, H-3′″″), 3.38 (m, H-4′″″), 3.70 (m, H-5′″″), 1.24 (d, J=6.1 Hz, 3H, H-6′″″); ¹³C-NMR (CD₃OD, 100 MHz): δ 38.56 (t, C-1), 30.75 (t, C-2), 79.28 (d, C-3), 39.50 (t, C-4), 141.90 (s, C-5), 122.62 (d, C-6), 33.17 (t, C-7), 32.77 (d, C-8), 51.71 (d, C-9), 38.03 (s, C-10), 21.94 (t, C-11), 40.83 (t, C-12), 41.82 (s, C-13), 57.74 (d, C-14), 32.77 (t, C-15), 82.43 (d, C-16), 65.03 (d, C-17), 16.82 (q, C-18), 19.85 (q, C-19), 41.15 (d, C-20), 16.13 (q, C-21), 113.98 (s, C-22), 31.39 (t, C-23), 28.97 (t, C-24), 34.99 (d, C-25), 76.01 (t, C-26), 17.29 (q, C-27), 3-O-glu: 100.48 (d, C-1′), 77.88 (d, C-2′), 77.95 (d, C-3′), 79.49 (d, C-4′), 76.67 (d, C-5′), 61.92 (t, C-6′), rha-(1→2): 102.34 (d, C-1″), 72.39 (d, C-2″), 72.39 (d, C-3″), 73.83 (d, C-4″), 69.78 (d, C-5″), 17.85 (q, C-6″), rha-(1→4)-rha-(1→4): 102.61 (d, C-1′″), 72.93 (d, C-2′″), 72.91 (d, C-3′″), 80.84 (d, C-4′″), 69.08 (d, C-5′″), 18.56 (q, C-6′″), 26-O-glu: 104.58 (d, C-1″″), 75.16 (d, C-2″″), 79.49 (d, C-3″″), 71.71 (d, C-4″″), 78.14 (d, C-5″″), 62.83 (t, C-6″″), rha-(1→4)-rha-(1→4): 103.16 (d, C-1′″″), 72.13 (d, C-2′″″), 72.39 (d, C-3′″″), 73.92 (d, C-4′″″), 70.44 (d, C-5′″″), 17.99 (q, C-6′″″); and ESI-MS (negative) [M−H]⁻ 1193.5 (calcd. for C₅₇H₉₃O₂₆ 1193.6).

### (3) Preparation and purification of peracetylated furost-5-ene-3,22,26-triol glycoside derivative of the present invention

The n-butanol soluble product (i.e. PK-1-1) was fractionated by gel column chromatography (Sephadex LH-20) and centrifugal partition chromatography (CPC) to obtain an active fractions (i.e., Fraction (Fr.) I-2-3). Sixty-one mg of this active fraction (Fr. I-2-3) in a 10-ml round bottom flask was reacted at 58° C. for 12 hours with 0.1 ml of acetic anhydride and 0.1 ml of pyridine. After evaporation under vacuum, the residue was purified on a silica gel column (230-400 mesh; 25 to 40% of ethyl acetate/toluene) to obtain 25 mg of pseudodichotomin peracetate (Compound 2).

Pseudodichotomin peracetate (Compound 2) is an amorphous powder, [α]_{D}²⁵: −30° (c 1.0, CHCl₃); IR vₘₐₓ cm⁻¹ (KBr): 2941 (m), 1752 (s), 1636 (w), 1435 (w), 1373 (m), 1224 (s), 1139 (m), 1042 (s); ¹H-NMR (CDCl₃, 400 MHz): δ 1.06 (dt, J=3.3, 13.1 Hz, H-1α), 1.83 (m, H-1β), 1.91 (m, H-2α), 1.52 (m, H-2β), 3.55 (m, H-3), 2.41 (dd, J=3.7, 12.8 Hz, H-4α), 2.25 (dd, J=11.0, 12.8 Hz, H-4 β), 5.36 (br d, J=4.4 Hz, H-6), 1.55 (m, H-7α), 2.01 (m, H-7β), 1.57 (m, H-8), 0.94 (dd, J=5.0, 10.7 Hz, H-9), 1.53 (m, H-11α), 1.49 (m, H-11β), 1.22 (m, H-12α), 1.77 (m, H-12β), 0.96 (m, H-14), 2.13 (m, H-15α), 1.38 (m, H-15β), 4.69 (ddd, J=5.6, 7.7, 9.8 Hz, H-16), 2.44 (br d, J=9.8 Hz, H-17), 0.64 (s, 3H, H-18), 0.98 (s, 3H, H-19), 1.54 (s, 3H, H-21), 2.06 (m, H-23), 2.04 (m, H-23), 1.22 (m, H-24), 1.53 (m, H-24), 1.68 (m, H-25), 3.27 (dd, J=6.0, 9.5 Hz, H-26), 3.67 (m, H-26), 0.85 (d, J=6.6 Hz, 3H, H-27), 3-O-glu: 4.54 (d, J=7.8 Hz, H-1′), 3.53 (dd, J=7.8, 9.4 Hz, H-2′), 5.26 (t, J=9.4 Hz, H-3′), 3.69 (t, J=9.5 Hz, H-4′), 3.58 (m, H-5′), 4.31 (dd, J=3.6, 12.4 Hz, H-6′), 4.45 (m, H-6′), rha-(1→2): 4.87 (d, J=1.3 Hz, H-1″), 5.01 (m, H-2″), 5.22 (dd, J=3.4, 10.1 Hz, H-3″), 5.02 (m, H-4″), 4.34 (m, H-5″), 1.16 (d, J=6.2 Hz, 3H, H-6″), rha-(1→4)-rha-(1→4): 4.73 (d, J=1.7 Hz, H-1′″), 5.08 (m, H-2′″), 5.15 (m, H-3′″), 3.58 (t, J=9.5 Hz, H-4′″), 3.79 (dq, J=6.1, 9.5 Hz, H-5′″), 1.26 (d, J=6.1 Hz, 3H, H-6′″), 26-O-glu: 4.43 (d, J=7.9 Hz, H-1″″), 4.96 (dd, J=7.9, 9.5 Hz, H-2″″), 5.16 (t, J=9.5 Hz, H-3″″), 5.05 (m, H-4″″), 3.65 (m, H-5″″), 4.10 (dd, J=2.2, 12.2 Hz, H-6″″), 4.23 (dd, J=4.6, 12.2 Hz, H-6″″), rha-(1→4)-rha-(1→4): 4.89 (d, J=1.6 Hz, H-1′″″), 5.03 (m, H-2′″″), 5.04 (m, H-3′″″), 5.03 (m, H-4′″″), 3.93 (dq, J=6.2, 9.5 Hz, H-5′″″), 1.17 (d, J=6.2 Hz, 3H, H-6′″″), 1.94˜2.11 (OAc groups); ¹³C-NMR (CDCl₃, 100 MHz.): δ 37.13 (t, C-1), 29.54 (t, C-2), 79.17 (d, C-3), 38.26 (t, C-4), 140.07 (s, C-5), 122.00 (d, C-6), 32.15 (t, C-7), 31.20 (d, C-8), 50.01 (d, C-9), 36.79 (s, C-10), 20.95 (t, C-11), 39.45 (t, C-12), 43.20 (s, C-13), 54.93 (d, C-14), 34.07 (t, C-15), 84.23 (d, C-16), 64.14 (d, C-17), 13.93 (q, C-18), 19.24 (q, C-19), 103.62 (s, C-20), 11.60 (q, C-21), 151.60 (s, C-22), 23.22 (t, C-23), 30.79 (t, C-24), 32.74 (d, C-25), 75.14 (t, C-26), 16.51 (q, C-27), 3-O-glu: 99.62 (d, C-1′), 76.30 (d, C-2′), 75.44 (d, C-3′), 77.78 (d, C-4′), 72.25 (d, C-5′), 62.14 (t, C-6′), rha-(1→2): 97.27 (d, C-1″), 70.01 (d, C-2″), 68.59 (d, C-3″), 71.70 (d, C-4″), 66.38 (d, C-5″), 17.28 (q, C-6″), rha-(1→4)-rha-(1→4): 99.47 (d, C-1′″), 70.96 (d, C-2′″), 68.68 (d, C-3′″), 79.07 (d, C-4′″), 68.50 (d, C-5′″), 17.72 (q, C-6′″), 26-O-glu: 100.96 (d, C-1″″), 71.30 (d, C-2″″), 72.86 (d, C-3″″), 68.50 (d, C-4″″), 71.70 (d, C-5″″), 61.98 (t, C-6″″), rha-(1→4)-rha-(1→4): 99.41 (d, C-1′″″), 70.08 (d, C-2′″″), 70.24 (d, C-3′″″), 70.85 (d, C-4′″″), 67.23 (d, C-5′″″), 17.17 (q, C-6′″″), 20.50˜20.90 and 169.20˜170.70 (OAc group); and FAB-MS (positive) [M+Na]⁺1787.7 (calcd. for C₈₅H₁₂₀O₃₉+Na 1787.7)°

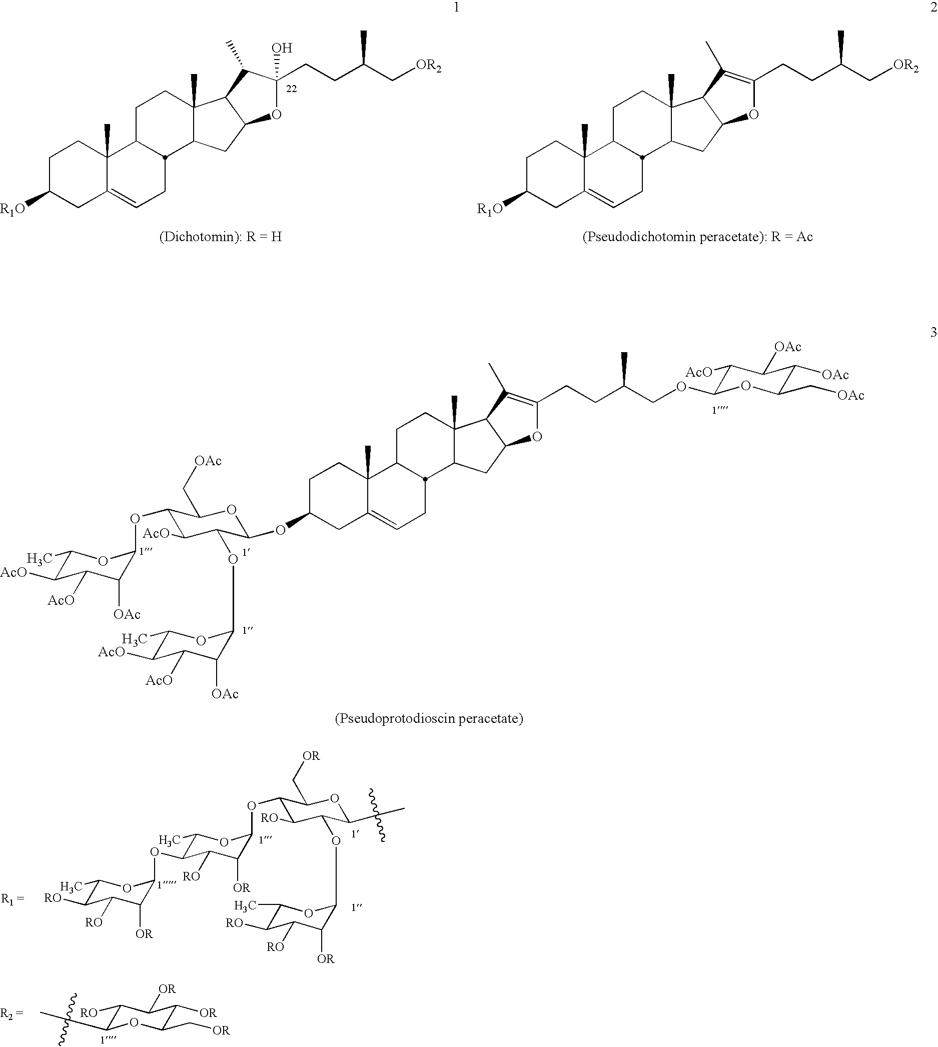

### (4) Isolation of furostanoid glycosides from Asparagus cochinchinensis (Lour.) Merr.

Fresh and ripe fruits of A. cochinchinensis (Lour.) Merr. without seeds (4.26 kg) were milled with a blender in 3 L of water. After centrifugation, the supernatant was concentrated to obtain 203 g of the water extract, which was dissolved in H₂O, was partitioned in sequence with ethyl acetate and n-butanol, to produce three fractions, i.e. ethyl acetate soluble fraction, n-butanol soluble fraction (18.2 g) and water soluble fraction. The n-butanol soluble fraction (17.0 g) was separated with a large-scale centrifugal partition chromatography (CPC) (Sanki Engineering (Kyoto), Model LLI) into four fractions (i.e., Fr. A, 4.8 g; Fr. B, 3.2 g; Fr. C, 2.1 g; and Fr. D, 7.0 g) based on their similarities.

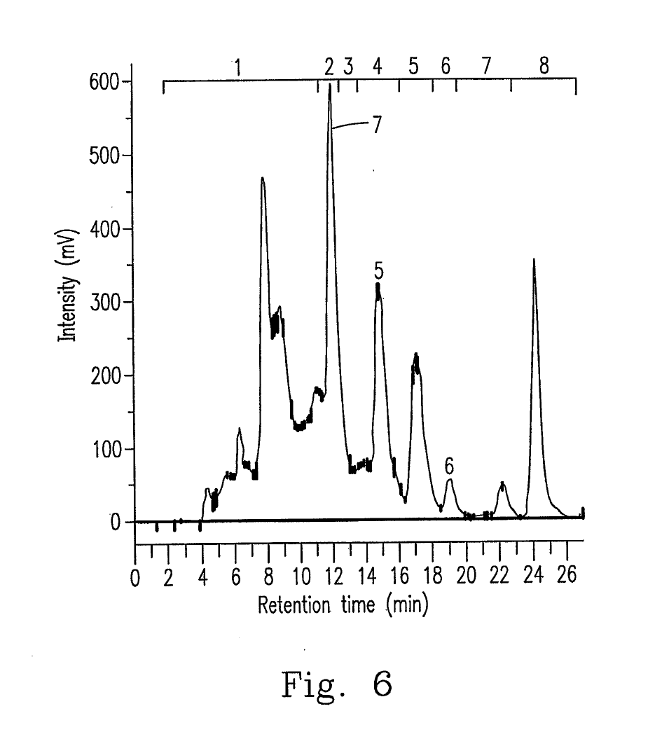

Fraction B (1.70 g) was further separated into four sub-fractions on Sephadex LH-20 with methanol as the solvent. Sub-fraction 2 (217 mg) was further purified with semi-preparative high performance liquid chromatography. The conditions used for HPLC were as follows: column, Merck, Purospher STAR RP-18e, 5 μm, 10×250 mm; delivery system: 70% methanol/water for 18 min, 70% to 90% methanol/water for 1 min (linear gradient), and 90% methanol/water for 8 min; flow rate: 3 ml/min; column temperature: 40° C.; evaporative light scattering detector (ELSD): 5% of eluent, gain 2; temperature: 40° C.; and pressure: 3.3 bar. Compound 5 (10 mg; retention time about 15.0 min), compound 6 (5 mg; retention time about 19.3 min), and compound 7 (30 mg; retention time about 12.3 min) were obtained (as shown in FIG. 6).

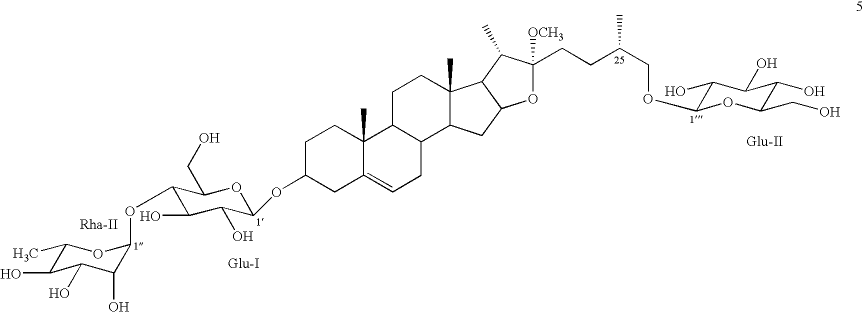

Compound 5 is 26-O-β-D-glucopyranosyl-22α-methoxy-(25S)-furost-5-ene-3β,26-diol 3-O-α-L-rhamnopyranosyl-(1→4)-β-D-glucopyranoside, shown above, is a white solid: [α]_{D}²⁷: −43.0° (c 1.0, MeOH); IR vₘₐₓ, cm⁻¹ (KBr): 3406, 2934, 1639, 1378, 1037; ¹H and ¹³C-NMR as shown in Tables 1 and 2; HMBC (CD₃OD, 400 MHz): H-4 to C-3, C-5; H-6 to C-4, C-8; H-15 to C-13, C-16; H-18 to C-12, C-13, C-14, C-17; H-19 to C-1, C-5, C-9, C-10; H-20 to C-13, C-17, C-21; H-21 to C-17, C-20, C-22; 22-OMe to C-22; H-27 to C-24, C-25, C-26; H-1′ to C-3; H-1′″ to C-26; and ESI-MS [M+H]⁺ m/z 917 (C₄₆H₇₆O₁₈+H).

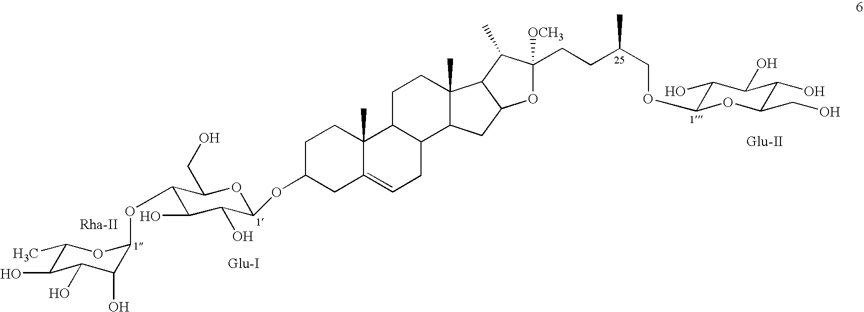

Compound 6 is 26-O-β-D-glucopyranosyl-22α-methoxy-(25R)-furost-5-ene-3β,26-diol 3-O-α-L-rhamnopyranosyl-(1→4)-β-D-glucopyranoside, shown above, is a white solid: [α]_{D}²⁷: −36.1° (c 0.7, MeOH); IR vₘₐₓ cm⁻¹ (KBr): 3397, 2934, 1652, 1379, 1035, 668; ¹H and ¹³C-NMR as shown in Tables 1 and 2; ESI-MS [M+H]⁺ m/z 917 (C₄₆H₇₆O₁₈+H).

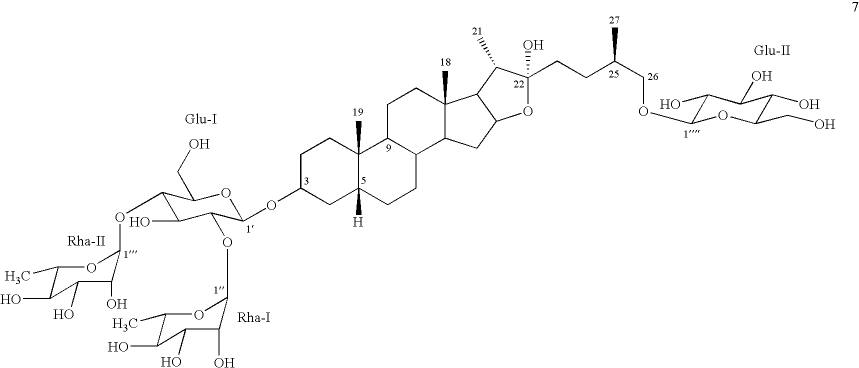

Compound 7 is 26-O-β-D-glucopyranosyl-22α-hydroxy-(25R)-furostane-3,26-diol 3-O-α-L-rhamnopyranosyl-(1→2)-[α-L-rhamnopyranosyl-(1→4)]-β-D-glucopyranoside, shown above, is a white solid: [α]_{D}²⁷: −58.0° (c 1.0, MeOH); IR vₘₐₓ cm⁻¹ (KBr): 3396, 2931, 1651, 1455, 1377, 1040, 910, 811, 668; ¹H and ¹³C-NMR data are shown in Tables 1 and 2; HMBC (CD₃OD, 400 MHz): H-6 to C-4, C-8; H-18 to C-12, C-13, C-14, C-17; H-19 to C-1, C-5, C-9, C-10; H-20 to C-13, C-17, C-21; H-21 to C-17, C-20, C-22; H-26 to C-24, C-25, C-27; H-27 to C-24, C-25, C-26; H-1′ to C-3; H-1″″ to C-26; and ESI-MS [M+K]⁺ m/z 1090 (C₅₁H₈₆O₂₂+K).

Tables 1 and 2, respectively, are ¹H-NMR and ¹³C-NMR data (CD₃OD) of the aglycone and glycone parts of compounds 5-7.

TABLE 1
¹H-NMR and ¹³C-NMR data of the aglycone parts of compounds 5-7

| | ¹H NMR (400 MHz) | | ¹³C NMR (100 MHz) | |
|---|---|---|---|---|
| Position | 5 & 6 | 7 | 5 & 6 | 7 |
| 1 | 1.07, 1.88 | 1.50, 1.80 | 39.7 t | 31.7 t |
| 2 | 1.29, 1.92 | 1.48, 1.76 | 30.7 t | 27.4 t |
| 3 | 3.58 m | 4.02 m | 79.9 d | 77.2 d |
| 4 | 2.27, 2.42 | 1.84, 1.88 | 38.5 t | 31.7 t |
| 5 | — | 1.61 | 142.0 s | 36.7 d |
| 6 | 5.36 | 1.44, 1.47 | 122.5 d | 27.5 t |
| 7 | 1.52, 2.02 | 1.21, 1.26 | 33.2 t | 27.6 t |
| 8 | 1.66 | 1.80 | 32.7 d | 38.1 d |
| 9 | 0.95 | 1.42 | 51.7 d | 41.5 d |
| 10 | — | — | 38.0 s | 36.1 s |
| 11 | 1.56 | 1.42 | 21.9 t | 22.0 d |
| 12 | 1.18, 1.78 | 1.18, 1.75 | 40.8 t | 41.3 t |
| 13 | — | — | 41.8 s | 42.2 s |
| 14 | 1.13 | 1.22 | 57.7 s | 57.6 d |
| 15 | 1.26, 1.98 | 1.22, 1.95 | 32.8 t | 32.7 t |
| 16 | 4.36 | 4.36 | 82.4 d | 82.5 d |
| 17 | 1.73 | 1.73 | 65.13 d (5) | 65.3 d |
| | | | 65.0 1d (6) | |
| 18 | 0.83 s | 0.79 s | 16.8 q | 17.0 q |
| 19 | 1.04 s | 1.00 s | 19.9 q | 24.2 q |
| 20 | 2.18 | 2.15 | 41.32 d (5) | 41.3 d |
| | | | 41.15 d (6) | |
| 21 | 0.99 d | 1.00 d | 16.18 q (5) | 16.3 q |
| | | | 16.13 q (6) | |
| 22 | — | — | 114.0 s | 113.9 s |
| 23 | 1.67, 1.77 (5) | 1.66, 1.78 | 31.41 t (5) | 31.4 t |
| | 1.62, 1.81 (6) | | 31.35 s (6) | |
| 24 | 1.25, 1.49 (5) | 1.23 | 28.9 t | 28.9 t |
| | 1.15, 1.62 (6) | | | |
| 25 | 1.72 | 1.73 | 35.06 d (5) | 35.1 d |
| | | | 34.98 (6) | |
| 26 | 3.34, 3.79 (5) | 3.34, 3.80 | 75.86 t (5) | 75.9 t |
| | 3.48, 3.73 (6) | | 76.00 5 (6) | |
| 27 | 0.95 s (5) | 0.96 s | 17.40 q (5) | 17.5 q |
| | 0.94 (6) | | 17.28 q (6) | |
| OMe | 3.13 | | 47.74 q (5) | |
| | | | 47.65 q (6) | |

TABLE 2
¹H-NMR and ¹³C-NMR data of the glycone parts of compounds 5-7

| | ¹H (400 MHz) | | ¹³C (100 MHz) | |
|---|---|---|---|---|
| Position | 5 & 6 | 7 | 5 & 6 | 7 |
| Glu-I | | | | |
| 1 | 4.39 (d, J = 7.8) | 4.38 (d, J = 7.7) | 102.3 d | 102.5 d |
| 2 | 3.19 | 3.47 | 75.1 d | 78.2 d |
| 3 | 3.45 | 3.58 | 76.7 d | 78.4 d |
| 4 | 3.52 | 3.52 | 79.6 d | 80.2 d |
| 5 | 3.35 | 3.30 | 76.7 d | 76.5 d |
| 6 | 3.64/3.82 | 3.64/3.77 | 62.0 t | 62.0 t |
| Rha-I | | | | |
| 1 | | 5.34 (d, J = 1.4) | | 101.7 d |
| 2 | | 3.91 | | 72.1 d |
| 3 | | 3.66 | | 72.2 d |
| 4 | | 3.40 | | 73.8 d |
| 5 | | 4.04 | | 69.8 d |
| 6 | | 1.21 (d, J = 6.2) | | 18.1 q |
| Rha-II | | | | |
| 1 | 4.84 brs | 4.82 | 102.9 d | 103.0 d |
| 2 | 3.82 | 3.85 | 72.4 d | 72.4 d |
| 3 | 3.62 | 3.62 | 72.1 d | 72.0 d |
| 4 | 3.42 | 3.42 | 73.7 d | 73.7 d |
| 5 | 3.96 | 3.91 | 70.6 d | 70.7 d |
| 6 | 1.26 (d, J = 6.2) | 1.25 (d, J = 6.2) | 17.8 q | 17.8 q |
| Glu-II | | | | |
| 1 | 4.23 (d, J = 7.8) | 4.22 (d, J = 7.7) | 104.6 d | 104.6 d |
| 2 | 3.19 | 3.18 | 75.2 d | 75.1 d |
| 3 | 3.35 | 3.35 | 76.7 d | 78.1 d |
| 4 | 3.35 | 3.27 | 71.7 d | 71.7 d |
| 5 | 3.28 | 3.27 | 78.1 d | 77.8 d |
| 6 | 3.66, 3.89 | 3.66, 3.87 | 62.8 t | 62.8 t |

### (5) Assay of L. chinensis Water Extract of the Present Invention Via Oral Administration

GP7TB rat liver cancer cells (10⁶ or 3×10⁶ cells) were subcutaneously injected into the backs of three 8-week old F344 female rats, each of which had a body weight of about 200 g. L. chinensis water extracts and a negative control, phosphate buffered saline (PBS), were fed to these rats by oral administration via gavage feeding. The dosage of L. chinensis water extract was 0.1 g per rat per day (corresponding to 0.5 g/kg), and oral administration via gavage feeding was performed for 20 or 40 days. Tumor cell growths were observed, and the tumor sizes were determined and recorded. In the 40-day experiment, no tumor growth was found in the L. chinensis water extract fed group, as shown in Table 3.

TABLE 3
Results of L. chinensis water extract by gavage feeding in rats

| | | Tumor size | Tumor size |
|---|---|---|---|
| | Drug | after 20 days | after 40 days |
| GP7TB/10⁶/ | PBS | 0.8 × 0.4 × 0.3 cm³ | 2.4 × 1.2 × 1.1 cm³ |
| S.C. | | | |
| GP7TB/10⁶/ | L. chinensis | 0.1 × 0.1 × 0.1 cm³ | ND |
| S.C. | water extract | | |
| GP7TB/ | L. chinensis | ND | ND |
| 3 × 10⁶/S.C. | water extract | | |

S.C.: subcutaneous injection;
ND: not detectable.

### (6) Single Dose Toxicity Tests of Compositions of the Present Invention

Normal SCID/CB17 female mice, 8-week old and each with body weight of about 20 g, were grouped into three groups of five mice each. PK-1-1 or PK-1-2 at a dose of 1 g/kg was intraperitoneally injected into the test groups of mice, and phosphate buffered saline (PBS) was injected into the control group. After two weeks, the growth of the mice seemed normally and the body weight gains were also normally. The tissue specimen of kidney, spleen, stomach, lung and brain were all normal.

### (7) Apoptosis induced by L. chinensis extracts PK-1-1 and PK-1-2 of the present invention

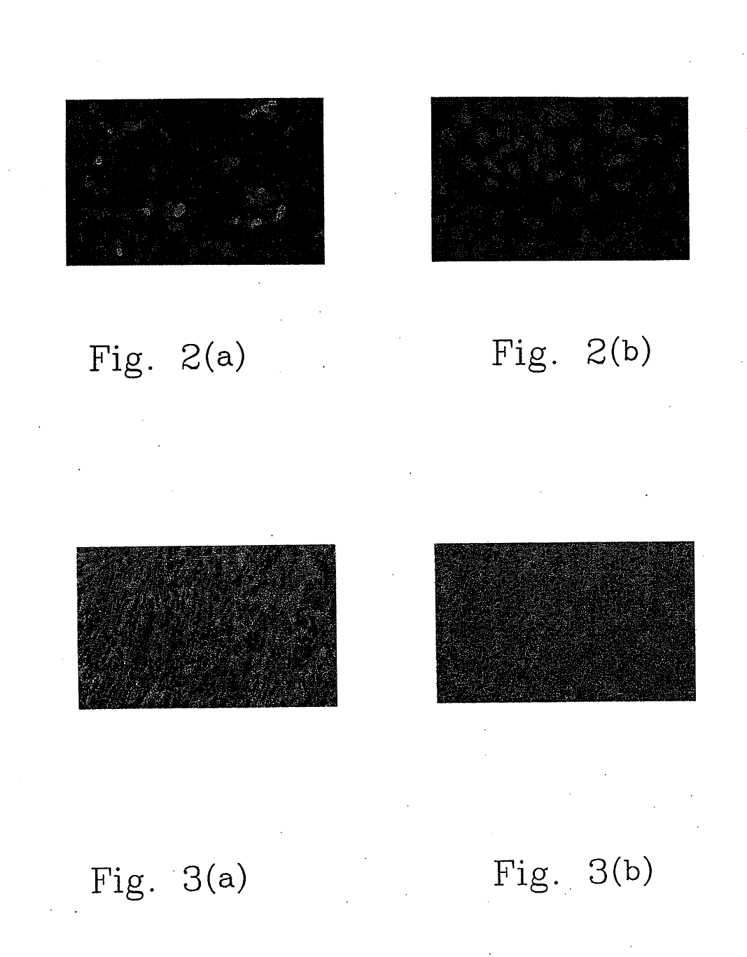

Rat liver cancer cells of GP7TB cell line were assayed with TαT-mediated dUTP nick-end labeling (TUNEL, Promega®) to evaluate whether the L. chinensis water extracts can induce apoptosis. The cells were grown on microscope slides and treated with 150 μg/ml of L. chinensis water extracts, PK-1-1 and PK-1-2, respectively, for 24 hours. After treatment with 4% formaldehyde and Triton X-100, the cells were reacted with TdT enzyme and stained with propidium iodide (PI). The observed yellow-green fluorescence represented the apoptotic cells. The results of treatments with L. chinensis water extracts PK-1-1 and PK-1-2 were shown, respectively, in FIGS. 2(a) and 2(b). Significant yellow-green fluorescence was seen in FIG. 2(a), indicating that PK-1-1 could induce the apoptosis of GP7TB cells.

### (8) Growth inhibition effects of L. chinensis extracts PK-1-1 and PK-17-1 and pure compound, dichotomin (PK-22-1), on liver cancer cell lines GP7TB, Huh-7 and HepG2

Growth inhibition and cytotoxic effects of L. chinensis extracts PK-1-1 and PK-17-1 were determined with cell survival assays using MTS (Promega®). Rat liver cancer cell line GP7TB and human liver cancer cell line Huh-7 and HepG2 at 10⁴ cells each were seeded in individual wells of a 96-well cultural plate. After overnight incubation, different concentrations of the extracts were added therein for 24 hours. Subsequently, the medium was discarded, and the medium with 20 μl/ml of MTS was added for 1 hour. Since MTS reagent contains 3-(4,5-dimethylhiazol-2-yl)-5-(3-carboxymethoxyphenyl)-2-(4-sulfophenyl)-2H-tetrazolium, which could be metabolized by NADH or NADPH dehydrogenase in living cells to a formazan product, cellular survival rates could be estimated from different readings at 490 nm. As shown in Tables 4 and 5, the extract PK-17-1 inhibited the growth of GP7TB cells at a concentration of 25 μg/ml. This extract had the same inhibition effect towards Huh-7 and HepG2 cells.

TABLE 4
MTS analysis of PK-1-1 and PK17-1
effects towards liver cancer cells

| | OD₄₉₀* | | | | | |
|---|---|---|---|---|---|---|
| Conc. | GP7TB | | Huh-7 | | HepG2 | |
| (μg/ml) | PK-1-1 | PK-17-1 | PK-1-1 | PK-17-1 | PK-1-1 | PK-17-1 |
| 0 | 1.403 | 1.181 | 0.676 | 0.51 | 0.765 | 0.669 |
| 25 | 1.39 | [symbol] | 0.597 | [symbol] | 0.762 | [symbol] |
| 50 | 1.374 | [symbol] | 0.595 | [symbol] | 0.702 | [symbol] |
| 100 | [symbol] | [symbol] | [symbol] | [symbol] | [symbol] | [symbol] |

*Optical density (OD) in Italic bold type means the cell morphology has changed.

TABLE 5
Growth inhibition of PK-1-1 and PK17-1 towards liver cancer cells

| | Cell growth (×10⁴ cells) | | | | | |
|---|---|---|---|---|---|---|
| Conc. | GP7TB | | Huh-7 | | HepG2 | |
| (μg/ml) | PK-1-1 | PK-17-1 | PK-1-1 | PK-17-1 | PK-1-1 | PK-17-1 |
| 0 | 17.2 | 17.5 | 7.25 | 8 | 8.25 | 10.25 |
| 25 | 17.2 | 14.2 | 8 | 3.5 | 7.5 | 10 |
| 50 | 16.5 | 3 | 6.5 | 1.75 | 5.75 | 1.75 |
| 100 | 14.7 | 2.75 | 3 | 1 | 4 | 1.5 |

The experiments were performed in triplicate and the results were performed by average calculation. As shown in Table 6, the 50% inhibition concentration (IC₅₀) value of dichotomin (PK-22-1) towards GP7TB cells was 1.65 M (1.97 g/ml), and the IC₅₀ value of the positive control, adriamycin, was 2.66 M (1.54 g/ml).

TABLE 6
Growth inhibition of different concentrations of dichotomin (PK-22-1)
and Adriamycin towards GP7TB cells

Amount of Cells (cells/ml)*

| Concentration (μg/ml) | PK-22-1 | Adriamycin |
|---|---|---|
| 0 | 2.06 × 10⁵ | 1.71 × 10⁵ |
| 1 | 1.30 × 10⁵ | 7.80 × 10⁴ |
| 2 | 4.60 × 10⁴ | 2.30 × 10⁴ |
| 4 | 2.00 × 10⁴ | 2.00 × 10⁴ |
| 6 | 1.10 × 10⁴ | 3.00 × 10⁴ |
| 8 | 5.00 × 10⁴ | 2.50 × 10⁴ |
| 10 | 3.00 × 10³ | 2.30 × 10⁴ |
| 12 | 1.60 × 10³ | 1.60 × 10⁴ |

*Mean of triplicate experiments

### (9) In Vivo Growth Inhibition of Rat Liver Tumor by Dichotomin (PK-22-1) of the Present Invention

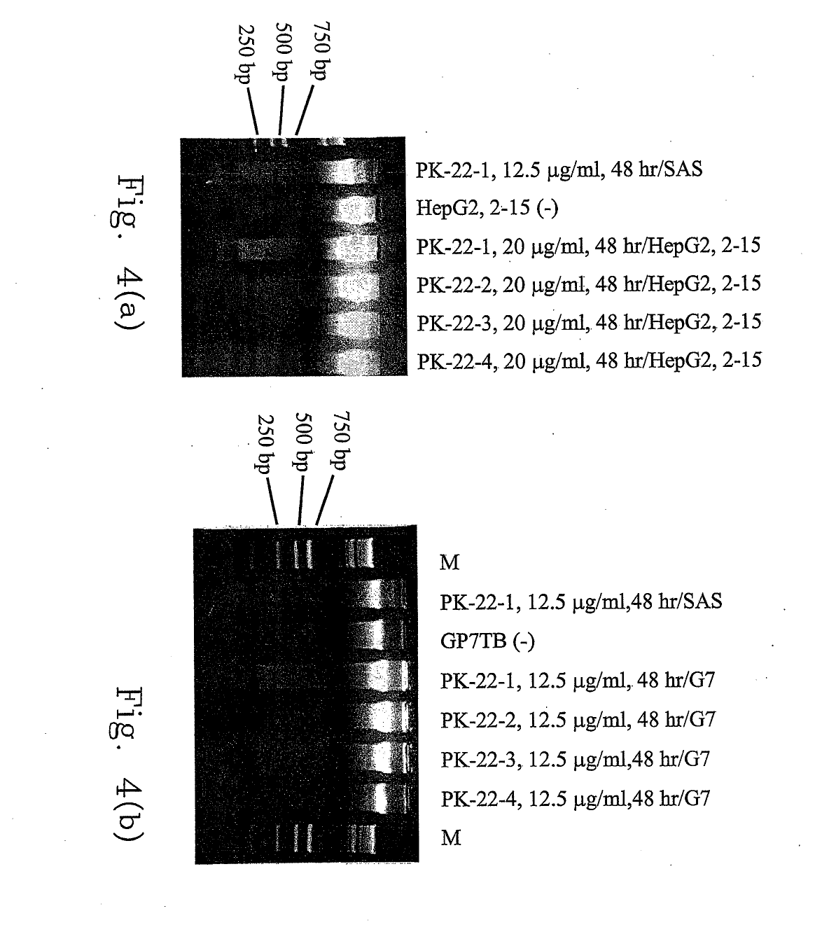

The back of each rat was injected subcutaneously with 3×10⁶ cells of GP7TB cells. The tumor grows up to 0.3 cm in diameter after about one week. A single dose of dichotomin (PK-22-1, 2.0 mg/kg) or the control buffer (PBS) was directly injected into the tumor once every day for 8 consecutive days. The tumor size was measured, and the cancer cell growth inhibition effects of the control group and dichotomin treated group were compared. As shown in Table 7, the tumor size of dichotomin injected rat was only half the size of the control group. Further, the cancer cells with dichotomin injection exhibited apoptosis, as shown in FIG. 3(b), while the cancer cells in the control group (PBS) still maintained the regular growth of pleomorphism, as shown in the pathological section of FIG. 3(a).

TABLE 7
Rat liver Cancer Cell Growth Inhibition Effects of dichotomin
(PK-22-1)

| | | | Growth |
|---|---|---|---|
| Drugs | Before injection (Day 0) | After injection (Day 8) | (folds) |
| PBS | 1.0 × 1.0 × 1.0 | 2.1 × 2.1 × 2.1 | 9.26 |
| | (0.52 cm³)* | (4.815 cm³) | |
| PK-22-1 | 0.65 × 0.65 × 0.65 | 1.1 × 1.1 × 1.1 | 4.87 |
| (2.0 mg/kg) | (0.142 cm³) | (0.692 cm³) | |

*tumor volume = 0.52 × length × width × height

### (10) Effects of Dichotomin (PK-22-1), Extracts with Dichotomin (PK-22-2 and PK-22-4), and Extracts without Dichotomin (PK-22-3) Extracted from L. chinensis on Nuclear DNA Fragmentation of GP7TB Cells

To understand the GP7TB cell growth inhibition mechanism of the active components and the extracts, GP7TB cells were treated with the extracts and dichotomin at a constant concentration of 12.5 μg/ml for 48 hours. The cellular DNA was extracted and analyzed with agarose gel electrophoresis. As shown in FIG. 4, the extracts containing dichotomin induced nuclear DNA fragmentation in GP7TB and HepG2 cells. Among the extracts, dichotomin (PK-22-1) showed the most notable effects. Therefore, it could be concluded that dichotomin could induce apoptosis of the abovementioned cell lines.

### (11) Ex-Vivo Anti-Tumor Experiment with Dichotomin (PK-22-1) and the Extract without Dichotomin (PK-22-3) of the Present Invention

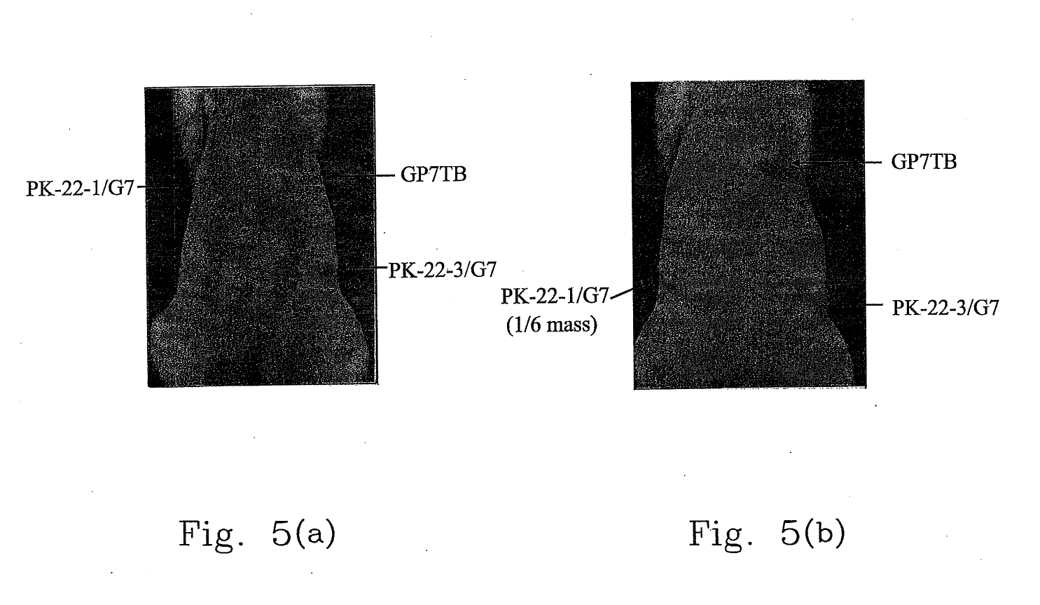

GP7TB cells (3×10⁶ cells each) were incubated in several 10-cm cultural dishes and treated with dichotomin (PK-22-1) or the extract without dichotomin (PK-22-3) at a concentration of 12.5 μg/ml for 24 hours. These cells were collected. GP7TB cells, PK-22-1-treated GP7TB cells, and PK-22-3-treated GP7TB cells (5×10⁶ cells each) were separately injected subcutaneously at three different locations on the back of each F344 rat. After three-week, six rats of the nine inoculated with dichotomin (PK-22-1)-treated GP7TB cells did not form any tumor, as shown in FIG. 5(a), while the other three rats inoculated with dichotomin (PK-22-1)-treated GP7TB cells had tumors of the sizes one sixth the sizes of the tumors arising from GP7TB cells (control group). The tumor sizes of rats inoculated with GP7TB cells treated with the extract PK-22-3 without dichotomin were the same as those of rats treated with GP7TB cells (control group).

### (12) Inhibition of Other Cancer Cells by Dichotomin (PK-22-1) of the Present Invention

Each suspension of various cancer cell lines was inoculated in a 96-well cultural plate and incubated at 37° C. under 5% carbon dioxide for 24 hours. Then, 100 μL of medium and 2 μl of different concentrations (100, 10, 1, 0.1 and 0.01 μM) of dichotomin (PK-22-1) were added therein. In addition, the cancer cells treated with a control drug, mitomycin, were incubated and treated in the same manner. After 72 hours of incubation, 20 μl of alamarBlue reagent was added into each well and the cells were incubated for another 6 hours. Cell densities were detected with a fluorescence detector, GENios equipped with a microreader, using an exciting wavelength of 530 nm and an emission wavelength of 590 nm. The IC₅₀ value means the concentration at which the experimental drug reduces the cell numbers by 50% at the end of the experiment. This value represents the inhibition activity of the test drugs.

Dichotomin was found to also have growth inhibition activity towards other cancer cells. As shown in Table 8, dichotomin had better inhibition activities towards the colon cancer cells and the lung cancer cells, as compared to the liver cancer cells.

TABLE 8
Inhibition of other cancer cell lines by dichotomin (PK-22-1)

| Compound | Cancer cell line | IC₅₀ (μM)* |
|---|---|---|
| PK-22-1 (experimental group) | Colon, HT-29 | 7.3 |
| | Liver, Hep 3B | 8.5 |
| | Lung, A549 | 0.84 |
| Mitomycin (control group) | Colon, HT-29 | 0.33 |
| | Liver, Hep 3B | 0.04 |
| | Lung, A549 | 0.16 |

*IC₅₀ is the lowest concentration reaching 50% inhibition.

Based on the abovementioned experimental results, furost-5-ene-3,22,26-triol glycoside compound clearly has the ability to promote apoptosis in various cancer cells, and therefore it can be used to prevent or treat various cancers in mammals and humans.

While the invention has been described using preferred embodiments, it is to be understood that the invention is not limited to the disclosed examples. On the contrary, one skilled in the art would appreciate that various modifications and variations are possible without departing from the scope of the invention as defined in the appended claims, which are to be accorded the broadest interpretation so as to encompass all such modifications and variations.

1. A pharmaceutical composition, comprising a furost-5-ene-3,22,26-triol glycoside having a structure shown in formula I, 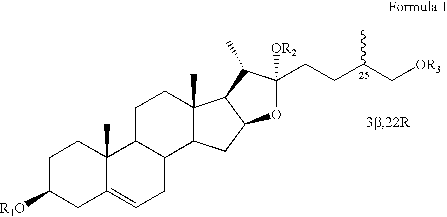
wherein R₁ is one selected from the group consisting of a hydrogen, a glucose, a rhamnose, a galactose, a xylose, an arabinose, a di-saccharide, a tetra-saccharide, a penta-saccharide, and a hexa-saccharide, wherein the di-saccharide, the tetra-saccharide, the penta-saccharide, and the hexa-saccharide are each composed of monosaccharides selected from the group consisting of glucose, rhamnose, galactose, xylose, and arabinose;
the stereo configuration at C-25 is either R (rectus) or S (sinister);
R₂ is one selected from the group consisting of hydrogen and a methyl group;
R₃ is one selected from the group consisting of hydrogen, glucose, rhamnose, galactose, xylose, and arabinose; and
a pharmaceutically acceptable carrier or excipient.

2-4. (canceled)

5. The pharmaceutical composition according to claim 1, wherein the furost-5-ene-3,22,26-triol glycoside is a (25R)-26-O-β-D-glucopyranosyl-22-hydroxy-5-ene-furostan-3β,26-diol-3-O-α-L-rhamnopyranosyl-(1→4)-α-L-rhamnopyranosyl-(1→4)-[α-L-rhamnopyranosyl-(1→2)]-β-D-glucopyranoside. (dichotomin).

6. The pharmaceutical composition according to claim 1, wherein the furost-5-ene-3,22,26-triol glycoside is a 26-O-β-D-glucopyranosyl-22α-methoxy-(25S)-furost-5-ene-3β,26-diol 3-O-α-L-rhamnopyranosyl-(1→4)-β-D-glucopyranoside.

7. The pharmaceutical composition according to claim 1, wherein the furost-5-ene-3,22,26-triol glycoside is a 26-O-β-D-glucopyranosyl-22α-methoxy-(25R)-furost-5-ene-3β,26-diol 3-O-α-L-rhamnopyranosyl-(1→4)-β-D-glucopyranoside.

8. The pharmaceutical composition according to claim 1, wherein the furost-5-ene-3,22,26-triol glycoside is a 26-O-β-D-glucopyranosyl-22α-hyroxy-(25R)-furost-5-ene-3β,26-diol 3-O-α-L-rhamnopyranosyl-(1→2)-α-L-rhamnopyranosyl-(1→4)-β-D-glucopyranoside.

9. The pharmaceutical composition according to claim 1, wherein the furost-5-ene-3,22,26-triol glycoside is extracted from a plant of the Livistona genus.

10. The pharmaceutical composition according to claim 1, wherein the furost-5-ene-3,22,26-triol glycoside is extracted form a plant of the Asparagus genus.

11. A method for preparing a pharmaceutical composition, comprising:
(a) providing a plant of genus Livistona or Asparagus; and
(b) extracting the plant with water or an aqueous solution to obtain a water extract containing a furost-5-ene-3,22,26-triol glycoside of a formula I.

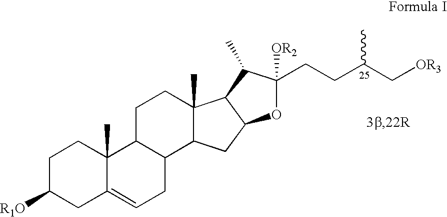

12. The method according to claim 11, further comprising a step of extracting the water extract with an organic solvent.

13. The method according to claim 12, wherein the organic solvent is n-butanol or ethyl acetate.

14. A method for preventing or treating a cancer, comprising:
administering to a subject an effective amount of the composition of claim 1.

15. The method according to claim 14, wherein the cancer is a liver cancer, a lung cancer, or a colon cancer.

16. The method according to claim 14, wherein the cancer is a liver cancer.

17. The method according to claim 14, wherein the subject is a mammal.

18. The method according to claim 17, wherein the mammal is a human.
